(19) United States
(12) Patent Application Publication
(10) Pub. No.: US 2006/0078619 A1
(43) Pub. Date: Apr. 13, 2006

(54) Method for the preparatin of paclitaxel solid dispersion by using the supercritical fluid process and paclitaxel solid dispersion prepared thereby

(75) Inventors: Jong-Soo Woo, KYUNGKI-DO (KR); Hyuk-Jin Kim, Kyungki-do (KR); Young-Hun Kim, Kyungki-do (KR)

Correspondence Address: SUGHRUE MION, PLLC, 2100 PENNSYLVANIA AVENUE, N.W., SUITE 800, WASHINGTON, DC, 20037, US

(73) Assignee: HANMI PHARM CO., LTD., KYUNGKI-DO (KR)

(21) Appl. No.: 10/534,353

(22) PCT Filed: Nov. 13, 2003
(86) PCT No.: PCT/KR03/02441

§ 371 Date: May 9, 2005

(30) Foreign Application Priority Data
Nov. 13, 2002 (KR) 10-2002-0070428

(51) Int. Cl. A61K 9/14; A61K 31/337

(52) U.S. Cl. 424/489; 514/449

(57) ABSTRACT

The present invention relates to a method for the preparation of paclitaxel solid dispersion by using the supercritical fluid process and paclitaxel solid dispersion prepared thereby, the paclitaxel solid dispersion being highly homogeneous and showing an improved solubility, thereby being effectively used for the preparation of paclitaxel injection and oral preparation having a high bioavailability.

### FIELD OF THE INVENTION

The present invention relates to a method for the preparation of a paclitaxel solid dispersion by a supercritical fluid process, a paclitaxel solid dispersion prepared thereby, and a pharmaceutical composition comprising same.

### BACKGROUND OF THE INVENTION

Paclitaxel is a powerful anticancer drug effective for treating ovarian, breast and lung cancers. Despite of its effectiveness, parclitaxel is sparingly soluble in water, which makes its bioavailability low, and therefroe, there have been several attempts to improve its solubility. For example, Korea Patent Publication No. 95-007872 discloses a liquid injection prepared by dissolving paclitaxel in an equal volume mixture of ethanol and Cremophor® EL, a surfactant derivative of castor oil. TAXOL® injection (Bristol-Myers Squibb) is prepared by dissolving 30 mg of paclitaxel in 5 ml of the 1:1 mixture of ethanol and Cremophor® EL. Since paclitaxel is usually administered at a dose of 175 mg/m² every 3 weeks (about 300 mg/60 kg), the amount of Cremophor® EL co-administered with the TAXOL® injection corresponding to 300 mg of paclitaxel becomes 25 ml. Cremophor® EL thus administered may induce a sensitive allergy reaction causing severe side effects, and accordingly, the TAXOL® injection is administered by drop-injecting over a prolonged time to avoid such adverse effects. Further, there is the inconvenience of having to take a preliminary drug therapy for a hypersensitive patient.

Therefore, several studies have been attempted to develop a formulation which can overcome the problems of the insolubility of paclitaxel and the excessive use of Cremorphor® EL. For example, International Patent Publication Nos. WO 00/01366 and WO 01/70220 teaches a liposome formulation of paclitaxel, but there are problems of a complicated preparative procedure and low stability. Further, International Patent Publication Nos. WO 01/26693, WO 00/53231 and WO 99/53951 describe an injection formulation which comprises a novel paclitaxel derivative and no Cremophor® EL, but the derivative carries the risk of triggering a toxicity problem.

For an oral formulation, it has been reported that paclitaxel can be absorbed only when administered in combination with P-glycoprotein (Leslie Z. Benet et al., J. Control. Rel. 39: 139-143, 1996). However, as paclitaxel must be soluble enough in water to be absorbed, the need to improve the paclitaxel's solubility has become a critical issue.

Thus, there has been an increasing demand for developing a paclitaxel injection formulation which can be easily prepared, contains a minimal amount of Cremophor® EL owing to improved the paclitaxel's solubility, and is non-toxic, as well as for an oral composition having an improved solubility of paclitaxel.

Meanwhile, a supercritical fluid is an incompressible fluid which exists at the temperature and pressure ranges beyond its critical points and has features that are uniquely different from those of conventional organic solvent. Namely, a supercritical fluid has the advantage properties of both liquid and gas, e.g., a high density close to that of a liquid, a low viscosity and high diffusion coefficient close to those of gas, and a very low surface tension.

Since the density of a supercritical fluid can be continuously changed from a sparse state like an ideal gas to a highly dense state like a liquid, its physicochemical properties at equilibrium (e.g., solubilizing power), mass transfer characteristics (e.g., viscosity, diffusion coefficient and thermal conductivity) and molecular clustering state of the fluid can be regulated. Therefore, by regulating the properties of a supercritical fluid, it is possible to obtain a solvent having properties which correspond to a combination of those of several solvents. Supercritical carbon dioxide, in particular, has a low critical temperature of 31.1° C. so that it can be used for a thermally unstable substance such as a protein drug. Further, since the supercritical carbon dioxide is nontoxic, incombustible, inexpensive and recyclable, it is environmentally friendly and can be advantageously used in a process of preparing medical products.

Many studies to take advantages of the unique features of supercritical fluid have recently been attempted in various fields, e.g., selective separation of a target material and analysis of materials extracted therewith fluid. Further, methods for obtaining crystal particles or microparticles using a supercritical fluid as a solvent or an anti-solvent have been actively studied.

The present inventors have endeavored to solve the paclitaxel's poor solubility in water and developed a method for the preparation of a paclitaxel solid dispersion by a supercritical fluid process. It has been found that a highly uniform nano-scale paclitaxel solid dispersion with an improved solubility can be prepared by changing the operating parameters of the supercritical fluid process, e.g., temperature and pressure, and additives used therewith.

### SUMMARY OF THE INVENTION

Accordingly, it is an object of the present invention to provide a method for preparing a highly uniform nano-scale paclitaxel solid dispersion with an improved solubility by the supercritical fluid process.

Another object of the present invention is to provide a paclitacel solid dispersion prepared by the above method and a pharmaceutical composition comprising same.

### BRIEF DESCRIPTION OF THE DRAWINGS

FIG. 1: a schematic procedure of the supercritical fluid process and a device thereof according to the present invention;

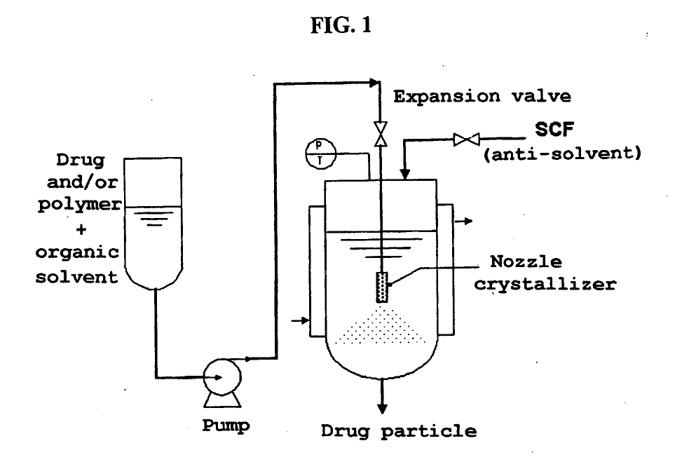

FIG. 2: a differential scanning calorimeter (DSC) scan of the inventive paclitaxel powder;

FIG. 3: a DSC scan of the paclitaxel formulation of Example 1;

FIG. 4: a scanning elecron microscopy (SEM) picture of the paclitaxel formulation of Example 1;

FIG. 5: a SEM picture of the paclitaxel formulation of Example 22.

### DETAILED DESCRIPTION OF THE INVENTION

In accordance with one aspect of the present invention, there is provided a method for preparing a highly uniform nano-scale paclitaxel solid dispersion which comprises the steps of spraying a mixture of paclitaxel and a pharmaceutically acceptable additive dissolved in a mixed organic solvent to a supercritical fluid to form particles of the mixture of paclitaxel and the pharmaceutically acceptable additive; and removing the organic solvent by washing the particles with the supercritical fluid.

In accordance with another aspect of the present invention, there is provided a highly uniform nano-scale paclitaxel solid dispersion prepared by the above method and a pharmaceutical composition comprising said paclitaxel solid dispersion which has an improved solubility.

The basic concept of the inventive method is to prepare a highly uniform nano-scale paclitaxel solid dispersion by mixing paclitaxel and a pharmaceutically acceptable additive in a suitable amount of a mixed organic solvent obtained by mixing two kinds of organic solvents in a proper ratio; spraying the resulting mixture into a reactor containing a supercritical fluid via a nozzle to obtain particles of a mixture of paclitaxel and the pharmaceutically acceptable additive; extractively removing the organic solvent by washing with freshly introduced supercritical fluid several times; and removing the supercritical fluid therefrom.

Particularly, the method for the preparation of a nano-scale highly uniform paclitaxel solid dispersion by the superficial fluid process of the present invention comprises the following steps:

1) preparing a mixture of paclitaxel and a pharmaceutically acceptable additive and dissolving them in a mixed organic solvent;

2) forming particles of the mixture of paclitaxel and the pharmaceutically acceptable additive by spraying the solution of the mixture of Step 1) to the supercritical fluid to bring into contact with each other;

3) removing the organic solvent by washing the particles with a fresh batch of the supercritical fluid; and

4) recovering the particle prepared thereby.

The above steps of the inventive method are each described in detail as follows.

(Step 1) Preparation of the Mixture of Paclitaxel and a Pharmaceutically Acceptable Additive

In Step 1), a mixture of paclitaxel and a pharmaceutically acceptable additive is prepared and dissolved them in a mixed organic solvent. Here, the pharmaceutically acceptable additive is added to enhance the degree of the crystallinity and solubility of paclitaxel, and includes, not but limited to, a hydrophilic polymer, a surfactant, an oily substance and so on.

The mixted solvent is prepared by mixing a 1^{st} organic solvent to dissolve paclitaxel and a 2^{nd} organic solvent to dissolve the additive, and then, paclitaxel and the additive were dissolved therein. At this time, it is preferable to mix the 1^{st} and the 2^{nd} organic solvents in a molar ratio of 7:3 to 5:5, more preferably about 6:4. The 1^{st} organic solvent used for dissolving paclitaxel includes, not but limited to, dichloromethane, chloroform, carbon tetrachloride, ethylacetate, N,N-dimethylformamide, DMSO and tetrahydrofuran, preferably dichloromethane. Further, the 2^{nd} organic solvent used for dissolving additives includes, not but limited to, ethanol, methanol and isopropanol, preferably ethanol. Since the above two organic solvents can easily mix with each other, a mixture thereof forms a homogenous solution.

The hydrophilic polymer employable as an additive in the present invention includes, not but limited to, hydroxymethylcellulose (HPMC), polyvinylpyrrolidone, hydroxypropylcellulose (HPC), hydroxyethylcellulose (HEC) and Eudragit, preferably hydroxypropylcellulose. The hydrophilic polymer may be suitably selected based on the solution's viscosity to be sprayed and its water solubility. For example, when a solid dispersion is not formed due to the presence of a excessive amount of a surfactant in the liquid phase during the supercritical fluid treatment, it is desirable to increase the amount of HPMC. It is preferable to employ the hydrophilic polymer in the amount ranging from 0.1 to 20 weight part, more preferably 1 to 10 weight part based on 1 weight part of paclitaxel.

In case of using the hydrophilic polymer as an additive, it is important to regulate the concentrations of the polymer and paclitaxel. Namely, when the concentration of the hydrophilic polymer is low, the amount of organic solvent increases resulting in lowering the preparation efficiency, and when it is high, the solution viscosity may become as high as to clog the spraying nozzle due to premature crystallization during spraying of the supercritical fluid. Accordingly, it is desirable that the amount of the hydrophilic polymer in the spray solution, on a solvent-free basis, is in the range of about 1 to 75% (w/w), more preferably 10 to 20% (w/w). In addition, as the diameter of the spraying nozzle inlet becomes smaller, it is desirable to lower the concentration of the solution.

Further, the surfactant employable as an additive in the present invention maybe:

\textcircled{1} a reaction product of a natural or hydrogenous plant oil and ethylene glycol, which is a polyoxyethylene glycolized natural or hydrogenous plant oil, e.g., a polyoxyethylene glycolized natural or hydrogenous castor oil (the name for sale: Cremophor® or HCO® (Nikkol));

\textcircled{2} polyoxyethylene-sorbitan-fatty acid ester, e.g., mono-, tri-lauryl ester, palmityl ester, stearyl ester and olely ester (the name for sale: Tween®, ICI);

\textcircled{3} polyoxyethylene stearic acid ester, e.g., polyoxyethylene stearic ester (the name for sale: Myrj®, ICI);

\textcircled{4} polyoxyethylene-polyoxypropylene block copolymer (the name for sale: Poloxamer, Pluronic® or Lutrol, BASF);

\textcircled{5} sodium dioctylsulfosuccinate or sodium lauryl sulfate;

\textcircled{6} phospholipid;
\textcircled{7} propylene glycol mono-fatty acid ester or propylene glycol di-fatty acid ester, e.g., propyleneglycol dicaprylate, propyleneglycol monocaprylate, propyleneglycol dilaurate, propyleneglycol isostearate, propyleneglycol monolaurate and propyleneglycol lisinorate, preferably propyleneglycol monolaurate;

\textcircled{8} trans-esterifying reaction product of natural plant oil triglyceride and polyalkylene polyol (the name for sale: Labrafil® M, Gattefosse);

\textcircled{9} mono-, di- or mono/di-glyceride, e.g., caprilic/capric acid mono- and di-glyceride (the name for sale: Imwitor®, Huls Am);

\textcircled{10} sorbitan fatty acid ester, e.g., sorbitan monolaurate, sorbitan monopalmitate and sorbitan monostearate (the name for sale: Span®, ICI);

\textcircled{11} fatty acid ester of ascorbic acid, e.g., ascorbyl plamitate; or

\textcircled{12} polyethylene glycol 660 hydroxystearate (the name for sale: Solutol® HS-15, BASF)

It is preferable to use the surfactant in the amount ranging from 0.1 to 60 weight part, more preferably 0.3 to 50 weight part based on 1 weight part of paclitaxel.

For the preparation of a paclitaxel solid dispersion for injection, it is preferable to use a non-ionic surfactant, and for oral, any surfactant.

An oily substance which may be further included in the mixture as an additive is a pharmaceutically acceptable oil capable of mixing well with a surfactant to form an emulsion in water. Representative examples of the oily substance are:

\textcircled{1} fatty acid triglyceride, e.g., a medium chain fatty acid triglyceride such as a fractionated coconut oil (the name for sale: Miglyol);

\textcircled{2} mono-, di- and mono/di-glyceride, e.g., a mono- and di-glyceride of oleic acid;

\textcircled{3} fatty acid ester of a monovalent alkanol, which is C₈ to C₂₀ fatty acid ester of C₂ to C₃ monovalent alkanol, e.g., isopropyl mirystate, isopropyl palmitate, ethyl linolate or ethyl olate;

\textcircled{4} natural plant oil or animal oil, e.g., corn oil, olive oil, soy bean oil and fish oil;

\textcircled{5} hydrocarbon, e.g., squalene and squalane;

\textcircled{6} tocopherol, e.g., tocopherol, tocopherol acetate, tocopherol succinate and polyethylene-1000-tocopherol succinate (TPGS); and

\textcircled{7} prophylene fatty acid ester, e.g., propyleneglycol monocaprylate, propyleneglycol dicaprylate and propyleneglycol monolaurate.

It is preferable to use an oily substance in the amount ranging from 0 to 10 weight part, more preferably 0 to 5 weight part based on 1 weight part of paclitaxel.

(Step 2) Formation of a Mixed Particle by Spraying the Solution Mixture to a Supercritical Fluid

In this step, particles of a mixture of parclitaxel and the additive is formed by spraying the solution mixture prepared in Step 1) to a supercritical fluid. The supercritical fluid employable in Step 2) includes, not but limited to, supercritical carbon dioxide, supercritical dinitrogen oxide (N₂O), supercritical trifluoromethane, supercritical propane, supercritical ethylene and supercritical xenon, preferably supercritical carbon dioxide.

After introducing a supercritical fluid into a stainless steel reactor, the reactor's inside is maintained at a supercritical state by increasing the temperature and pressure over the critical points of the fluid and kept at an equilibrated supercritical state. For example, in case of using carbon dioxide having a critical temperature of 31.06° C. and a critical pressure of 73.8 bar as a supercritical fluid, it is needed to increase the reactor's temperature and pressure over said critical temperature and pressure, e.g., temperature ranging from 35 to 70° C. and pressure ranging from 80 to 200 bar, more preferably 40 to 60° C. and 100 to 150 bar. It is desirable to introduce the fluid using a syringe pump during for the pressurization to check the exact amount of the fluid injected, and a circular thermostat or an automatic temperature regulator to maintain a constant temperature.

When the reactor's inside reaches an equilibrated supercritical state, the solution containing the mixture of paclitaxel and the additive prepared in Step 1) is injected into the reactor at a constant rate using a liquid pump through a nozzle. At this time, in order to prevent the nozzle from clogging, it is desirable to inject a small amount of the blank solvent (the mixture of the 1^{st} and 2^{nd} organic solvents mixed in the ratio as described in Step 1), e.g., 3 to 4 ml, before injecting the solution. As the amount of the blank solvent injected increases, the time required for washing the reactor with a supercritical fluid may become longer.

After the solution injected into the reactor via a nozzle is sprayed, as the organic solvent of the sprayed solution blends rapidly with the supercritical fluid, the paclitaxel and additive become hypersaturated and precipitate, generating crystal particles.

In order to prevent the supercritical fluid in the reactor from being saturated during the solution injection, fresh supercritical fluid may further be introduced into the reactor.

(Step 3) Removal of the Organic Solvent Using a Supercritical Fluid

After the solution mixture is completely sprayed into the reactor, a washing step is conducted by incorporating a fresh batch of supercritical fluid into the reactor to remove the organic solvent from the particles generated. In the above step, a portion of the supercritical fluid is discharged via an outlet at a rate which is the same as the injection rate to continuously maintain the reactor pressure. At this time, a back pressure regulator is connected to the outlet to constantly maintain the reactor pressure by regulating the discharging rate. Two overlapping membrane filters having a pore size of 0.45 μm are installed at the outlet to hold the particles within the reactor.

If the organic solvent still remains, when the temperature and pressure are lowered to harvest the particles, the organic solvent may dissolve the particles again, resulting in the formation of coagulation. Therefore, the washing step must be carried out thoroughly to remove the last trace of the organic solvent. The amount of supercritical fluid for washing may differ according to the amount of organic solvent used and the reactor size. For example, it is preferable to use about 50 to 150 ml of supercritical fluid in case the reactor volume is 92.4 cm³.

(Step 4) Recovery of Particles

When the washing step is completed, the supply of supercritical fluid into the reactor is stopped and the supercritical fluid is discharged from the reactor. At this time, rapid discharge of the supercritical fluid may damage the particles, and accordingly, it is preferable to discharge gradually. After the supercritical fluid in the reactor is completely removed, the particles are recovered from the reactor's wall or bottom.

The particles thus recovered have a diameter ranging from 0.5 to 3 μm, and form a highly uniform spherical solid dispersion. The result of analyzing the thermochemical properties of the inventive solid dispersion with a differential scanning calorimeter (DSC) shows that while a paclitaxel powder shows a strong endothermic peak at around 156° C., the paclitaxel solid dispersion of the present invention does not show any endothermic peak. Accordingly, it has been conformed that the paclitaxel solid dispersion of the present invention is a highly uniform nano-scale solid dispersion having an altered molecular arrangement.

As described above, the present invention provides a method for obtaining crystal particles using a supercritical fluid as an anti-solvent. This method can be applicable when the solubilities of a polymer and drug to a supercritical fluid are sufficiently low. When the polymer and drug are dissolved in a conventional organic solvent which is completely miscible with a supercritical fluid and then the resulting mixture is sprayed within the supercritical fluid via a nozzle, the organic solvent will mix with the supercritical fluid as its solubility to the polymer and drug becomes reduced and the hypersaturated polymer and drug will precipitate out, resulting in the formation of crystalline particles. Here, it is possible to control the particle's properties by regulating the extracting rate of the organic solvent through manipulating the kind of the solution to be sprayed and the supercritical fluid's temperature and pressure, and therefore, it is possible to prepare a highly uniform nano-scale paclitaxel solid dispersion having an improved solubility.

The saturation solubility of the highly uniform nano-scale paclitaxel solid dispersion prepared by the supercritical fluid process of the present invention is several thousands-fold higher than that of a conventional paclitaxel powder (See Table 24).

Accordingly, since the inventive paclitaxel solid dispersion having an improved solubility is capable of dissolving easily in water in the presence of no or a small amount of Cremophor EL, it can be effectively used for the preparation of an injection or oral formulation of paclitaxel.

Further, the injection formulation comprising the inventive paclitaxel solid dispersion may have the advantage that it does not induce any adverse side effect because of the minimal amount of Cremophor EL used. Besides, since the injection formulation of the present invention does not form crystals such as a paclitaxel's precipitate even at a paclitaxel concentration which is higher than the conventional dilution concentration used for actual clinical administration, i.e., 0.3 to 1.2 mg/ml, it is possible to reduce the time for administration.

Accordingly, the present invention also provides a pharmaceutical composition comprising the paclitaxel solid dispersion as an effective ingredient in combination with pharmaceutically acceptable carriers, excipients or additives. The inventive pharmaceutical composition may be formulated in the form of oral or parenteral administration according to any one of the conventional procedures. The formulation for oral administration may be in the form of a tablet, pill, soft and hard gelatin capsule, solution, suspension, emulsion, syrup, granule and the like. These formulations may additionally include diluents (e.g., lactose, dextrose, sucrose, mannitol, sorbitol, cellulose and/or glycine), lubricating agents (e.g., silica, talc, stearic acid and magnesium or calcium salt thereof and/or polyethylene glycol) and the like. The tablet may also includes bonding agents such as magnesium aluminum silicate, starch paste, gelatin, tracagans, methylcellulose, sodium carboxymethylcellulose and/or polyvinylpyrrolidone; disintegrating agents such as starch, agar, alginic acid or sodium salt thereof or its equivalent mixture; and/or absorbing agents, coloring agents, flavoring agents and sweetening agents. The inventive formulations may be prepared according to any one of the conventional procedures for mixing, granulating or coating well known in the art. Further, the representative parenteral formulation is an injection. It is preferable to prepare the injection in the form of an isotonic aqueous solution or a suspension.

The pharmaceutical formulations of the present invention may be autoclaved and/or additionally include additives such as preservatives, stabilizing agents, hydrating agents, emulsifying agents, salts for regulating osmotic pressure and/or buffering agents and other therapeutically effective substances.

Paclitaxel as an effective ingredient may be administered in a single dose or in divided doses by oral or parenteral route.

The following Reference Examples, Test Examples and Formulation Examples are intended to further illustrate the present invention without limiting its scope.

### EXAMPLE 1

### Preparation of a Highly Uniform Nano-Scale Paclitaxel Solid Dispersion by Supercritical Fluid Process 1

In order to prepare a pacliitaxel solid dispersion for injection by the inventive supercritical fluid process, a mixture of paclitaxel and pharmaceutically acceptable additives for spraying was prepared using the constituents shown in Table 1.

TABLE 1

| Constituent | Weight (g) |
|---|---|
| Paclitaxel | 1 |
| HPMC 2910 | 7 |
| Myrj 52 | 25 |
| Solutol | 0.5 |
| Ascorbil palmitate | 0.5 |
| d-α-Tocopherol | 3 |
| Dichloromethane | 420 |
| Ethanol | 280 |

First, dichloromethane and ethanol were mixed by agitating, and then, paclitaxel (Hanmi Pharm. Co., Ltd.) and pharmaceutically acceptable additives; hydroxypropyl methyl cellulose (HPMC) 2910 (Shin-Etsu) as a hydrophilic polymer, Myrj 52 (ICI) as a surfactant, solutol (BASF), ascorbil palmitate (Roche) and d-α-tocopherol as fatty constituents (Roche); were added thereto to prepare a solution mixture while agitating.

The interior of a reactor (internal diameter of 2.9 cm, height of 14 cm, volume of 92.4 cm³; Greentek21) was adjusted to 40° C., liquid carbon dioxide was pressurized therein by using a syringe pump (Isco®, model 260D) to charge the reactor pressure to 103 bar, and then, the temperature and pressure were maintained until an equilibrated supercritical carbon dioxide environment was obtained.

Then, about 2 ml of a blank solvent (dichloromethane+ethanol [3:2 (v/v)]) was injected through a nozzle into the reactor to prevent the nozzle from clogging. The solvent mixture then was injected into the reactor using a small liquid pump (NP-AX-15, NIHON SEIMITSU KAGAKU CO., LTD, Japan). As the injected mixture was sprayed via a capillary nozzle in the reactor's inside, a highly uniform solid dispersion was formed under the supercritical state. To prevent the supercritical carbon dioxide in the reactor from saturating simultaneously with the injection of the mixture, a measured amount of carbon dioxide corresponding to the injected amount was discharged from the reactor at the same rate via an outlet as the fresh carbon dioxide was injected into the reactor at a rate of 10 ml/min.

When injection of the solution mixture was complete, the organic solvent dissolved in the supercritical carbon dioxide was removed by supercritical carbon dioxide washing; carbon dioxide was discharged from the reactor at a rate equal to the injection rate via an outlet using a back pressure regulator (TESCOM®, model 26-1723-24-194) while fresh carbon dioxide was injected into the reactor at a rate of 7 ml/min. Two overlapping membrane filters having a pore size of 0.45 μm were attached to the outlet to keep the particles within the reactor. Such washing was conducted using a total of 100 ml of carbon dioxide.

After the particles were completely washed, the carbon dioxide within the reactor was discharged at a rate of 10 ml/min, and then, a highly uniform nano-scale paclitaxel solid dispersion for injection was recovered from the reactor's wall and bottom.

### EXAMPLE 2

### Preparation of a Highly Uniform Nano-Scale Paclitaxel Solid Dispersion by Supercritical Fluid Process 2

A highly uniform nano-scale pacliitaxel solid dispersion for injection was prepared using the constituents shown in Table 2 according to the same supercritical fluid process as described in Example 1 except that polyvinylpyrrolidone K-30 (BASF) was employed as a polymer, and the supercritical operation was carried out under the condition of 40° C. and 103 bar.

TABLE 2

| Constituent | Weight (g) |
|---|---|
| Paclitaxel | 1 |
| Polyvinylpyrrolidone K-30 | 5 |
| Myrj 52 | 25 |
| Solutol | 0.75 |
| Ascorbil palmitate | 0.5 |
| d-α-Tocopherol | 3 |
| Dichloromethane | 400 |
| Ethanol | 268 |

### EXAMPLE 3

### Preparation of a Highly Uniform Nano-Scale Paclitaxel Solid Dispersion by Supercritical Fluid Process 3

A highly uniform nano-scale paciliitaxel solid dispersion for injection was prepared using the constituents shown in Table 3 according to the same supercritical fluid process as described in Example 1 except that Tween 80 (ICI) was employed as a surfactant, and the supercritical operation was carried out under the condition of 43° C. and 136 bar.

TABLE 3

| Constituent | Weight (g) |
|---|---|
| Paclitaxel | 1 |
| HPMC 2910 | 7 |
| Myrj 52 | 10 |
| Solutol | 0.5 |
| Tween 80 | 20 |
| d-α-Tocopherol | 3 |
| Dichloromethane | 470 |
| Ethanol | 315 |

### EXAMPLE 4

### Preparation of a Highly Uniform Nano-Scale Paclitaxel Solid Dispersion by Supercritical Fluid Process 4

A highly uniform nano-scale pacliitaxel solid dispersion for injection was prepared using the constituents shown in Table 4 according to the same supercritical fluid process as described in Example 1 except that Fluronic L44 (BASF) was employed as a surfactant, and the supercritical operation was carried out under the condition of 45° C. and 103 bar.

TABLE 4

| Constituent | Weight (g) |
|---|---|
| Paclitaxel | 1 |
| HPMC 2910 | 7 |
| Fluronic L-44 | 10 |
| Solutol | 0.5 |
| Myrj 52 | 15 |
| d-α-Tocopherol | 3 |
| Dichloromethane | 410 |
| Ethanol | 280 |

### EXAMPLE 5

### Preparation of a Highly Uniform Nano-Scale Paclitaxel Solid Dispersion by Supercritical Fluid Process 5

A highly uniform nano-scale pacliitaxel solid dispersion for injection was prepared using the constituents shown in Table 5 according to the same supercritical fluid process as described in Example 1 except that HPC(Shin-Etsu) as a polymer and Poloxamer 188 (BA SF) as a surfactant were employed, and the supercritical operation was carried out under the condition of 45° C. and 103 bar.

TABLE 5

| Constituent | Weight (g) |
|---|---|
| Paclitaxel | 1 |
| HPC | 7 |
| Poloxamer 188 | 10 |
| Solutol | 0.5 |
| Myrj 52 | 15 |
| d-α-Tocopherol | 3 |
| Dichloromethane | 416 |
| Ethanol | 280 |

### EXAMPLE 6

### Preparation of a Highly Uniform Nano-Scale Paclitaxel Solid Dispersion by Supercritical Fluid Process 6

A highly uniform nano-scale pacliitaxel solid dispersion for injection was prepared using the constituents shown in Table 6 according to the same supercritical fluid process as described in Example 1 except that Labrapil 1944 (Gattefosse) was employed as a surfactant, and the supercritical operation was carried out under the condition of 45° C. and 103 bar.

TABLE 6

| Constituent | Weight (g) |
|---|---|
| Paclitaxel | 1 |
| HPMC 2910 | 7 |
| Labrapil 1944 | 10 |
| Myrj 52 | 15 |
| d-α-Tocopherol | 3 |
| Dichloromethane | 410 |
| Ethanol | 270 |

### EXAMPLE 7

### Preparation of a Highly Uniform Nano-Scale Paclitaxel Solid Dispersion by Supercritical Fluid Process 7

A highly uniform nano-scale pacliitaxel solid dispersion for injection was prepared using the constituents shown in Table 7 according to the same supercritical fluid process as described in Example 1 except that Tween 20 and Tween 80 (ICI) were employed as a surfactant, and the supercritical operation was carried out under the condition of 45° C. and 103 bar.

TABLE 7

| Constituent | Weight (g) |
|---|---|
| Paclitaxel | 1 |
| HPMC 2910 | 7 |
| Tween 80 | 15 |
| Tween 20 | 10 |
| d-α-Tocopherol | 1 |
| Dichloromethane | 388 |
| Ethanol | 258 |

### EXAMPLE 8

### Preparation of a Highly Uniform Nano-Scale Paclitaxel Solid Dispersion by Supercritical Fluid Process 8

A highly uniform nano-scale pacliitaxel solid dispersion for injection was prepared using the constituents shown in Table 8 according to the same supercritical fluid process as described in Example 1 except that Span 60 (ICI) was employed as a surfactant, and the supercritical operation was carried out under the condition of 45° C. and 103 bar.

TABLE 8

| Constituent | Weight (g) |
|---|---|
| Paclitaxel | 1 |
| HPMC 2910 | 7 |
| Span 60 | 10 |
| Myrj 52 | 15 |
| Dichloromethane | 376 |
| Ethanol | 250 |

### EXAMPLE 9

### Preparation of a Highly Uniform Nano-Scale Paclitaxel Solid Dispersion by Supercritical Fluid Process 9

A highly uniform nano-scale pacliitaxel solid dispersion for injection was prepared using the constituents shown in Table 9 according to the same supercritical fluid process as described in Example 1 except that Tween 80 (ICI) and PEG 20,000 (Union Carbide) were employed as a surfactant, and the supercritical operation was carried out under the condition of 40° C. and 103 bar.

TABLE 9

| Constituent | Weight (g) |
|---|---|
| Paclitaxel | 1 |
| HPMC 2910 | 5 |
| Tween 80 | 1.5 |
| PEG 20,000 | 0.1 |
| Ascorbil palmitate | 0.1 |
| d-α-Tocopherol | 0.2 |
| Dichloromethane | 90 |
| Ethanol | 60 |

### EXAMPLE 10

### Preparation of a Highly Uniform Nano-Scale Paclitaxel Solid Dispersion by Supercritical Fluid Process 10

A highly uniform nano-scale pacliitaxel solid dispersion for injection was prepared using the constituents shown in Table 10 according to the same supercritical fluid process as described in Example 1 except that propylene glycol (Nikkol) was employed as a surfactant, and the supercritical operation was carried out under the condition of 40° C. and 103 bar.

TABLE 10

| Constituent | Weight (g) |
|---|---|
| Paclitaxel | 1 |
| HPMC 2910 | 7 |
| Myrj 52 | 2.5 |
| Propylene glycol | 1 |
| Ascorbil palmitate | 0.1 |
| d-α-Tocopherol | 0.2 |
| Dichloromethane | 134 |
| Ethanol | 90 |

### EXAMPLE 11

### Preparation of a Highly Uniform Nano-Scale Paclitaxel Solid Dispersion by Supercritical Fluid Process 11

A highly uniform nano-scale pacliitaxel solid dispersion for injection was prepared using the constituents shown in Table 11 according to the same supercritical fluid process as described in Example 1 except that dl-α-tocopheryl acetate (Roche) was employed as a fatty constituent, and the supercritical operation was carried out under the condition of 40° C. and 103 bar.

TABLE 11

| Constituent | Weight (g) |
|---|---|
| Paclitaxel | 1 |
| HPMC 2910 | 5 |
| Myrj 52 | 5 |
| Solutol | 0.2 |
| Ascorbil palmitate | 0.2 |
| dl-α-Tocopheryl acetate | 0.4 |
| Dichloromethane | 134 |
| Ethanol | 90 |

### EXAMPLE 12

### Preparation of a Highly Uniform Nano-Scale Paclitaxel Solid Dispersion by Supercritical Fluid Process 12

A highly uniform nano-scale pacliitaxel solid dispersion for injection was prepared using the constituents shown in Table 12 according to the same supercritical fluid process as described in Example 1 except that ethyl linoleate (Aldrich) was employed as a surfactant, and the supercritical operation was carried out under the condition of 45° C. and 103 bar.

TABLE 12

| Constituent | Weight (g) |
|---|---|
| Paclitaxel | 1 |
| HPMC 2910 | 3 |
| Ethyl linoleate | 1 |
| Solutol | 0.2 |
| Myrj 52 | 7 |
| Ascorbil palmitate | 0.5 |
| d-α-Tocopherol | 1 |
| Dichloromethane | 156 |
| Ethanol | 104 |

### EXAMPLE 13

### Preparation of a Highly Uniform Nano-Scale Paclitaxel Solid Dispersion by Supercritical Fluid Process 13

A highly uniform nano-scale pacliitaxel solid dispersion for injection was prepared using the constituents shown in Table 13 according to the same supercritical fluid process as described in Example 1 except that propyleneglycol monocaprylate (PGMC, Nikkol) was employed as a surfactant, and the supercritical operation was carried out under the condition of 40° C. and 103 bar.

TABLE 13

| Constituent | Weight (g) |
|---|---|
| Paclitaxel | 1 |
| HPMC 2910 | 3 |
| PGMC | 0.5 |
| Solutol | 0.2 |
| Myrj 52 | 7 |
| Ascorbil palmitate | 0.5 |
| d-α-Tocopherol | 1 |
| Dichloromethane | 150 |
| Ethanol | 100 |

### EXAMPLE 14

### Preparation of a Highly Uniform Nano-Scale Paclitaxel Solid Dispersion by Supercritical Fluid Process 14

A highly uniform nano-scale pacliitaxel solid dispersion for injection was prepared using the constituents shown in Table 14 according to the same supercritical fluid process as described in Example 1 except that Myrj 45 (ICI) was employed as a surfactant, and the supercritical operation was carried out under the condition of 40° C. and 103 bar.

TABLE 14

| Constituent | Weight (g) |
|---|---|
| Paclitaxel | 1 |
| HPMC 2910 | 7 |
| Solutol | 0.75 |
| Myrj 45 | 25 |
| Ascorbil palmitate | 0.5 |
| d-α-Tocopherol | 3 |
| Dichloromethane | 424 |
| Ethanol | 280 |

### EXAMPLE 15

### Preparation of a Highly Uniform Nano-Scale Paclitaxel Solid Dispersion by Supercritical Fluid Process 15

A highly uniform nano-scale pacliitaxel solid dispersion for injection was prepared using the constituents shown in Table 15 according to the same supercritical fluid process as described in Example 1 except that Myrj 59 (ICI) was employed as a surfactant, and the supercritical operation was carried out under the condition of 40° C. and 103 bar.

TABLE 15

| Constituent | Weight (g) |
|---|---|
| Paclitaxel | 1 |
| HPMC 2910 | 7 |
| Solutol | 0.75 |
| Myrj 59 | 25 |
| Ascorbil palmitate | 0.5 |
| d-α-Tocopherol | 3 |
| Dichloromethane | 424 |
| Ethanol | 280 |

### EXAMPLE 16

### Preparation of a Highly Uniform Nano-Scale Paclitaxel Solid Dispersion by Supercritical Fluid Process 16

A highly uniform nano-scale pacliitaxel solid dispersion for injection was prepared using the constituents shown in Table 16 according to the same supercritical fluid process as described in Example 1 except that Brij 35 (ICI) was employed as a surfactant, and the supercritical operation was carried out under the condition of 40° C. and 103 bar.

TABLE 16

| Constituent | Weight (g) |
|---|---|
| Pclitaxel | 1 |
| HPMC 2910 | 7 |
| Solutol | 0.75 |
| Brij 35 | 25 |
| Ascorbil palmitate | 0.5 |
| d-α-Tocopherol | 3 |
| Dichloromethane | 424 |
| Ethanol | 280 |

### EXAMPLE 17

### Preparation of a Highly Uniform Nano-Scale Paclitaxel Solid Dispersion by Supercritical Fluid Process 17

A highly uniform nano-scale pacliitaxel solid dispersion for injection was prepared using the constituents shown in Table 17 according to the same supercritical fluid process as described in Example 1 except that Poloxamer 188 (BASF) was employed as a surfactant, and the supercritical operation was carried out under the condition of 45° C. and 103 bar.

TABLE 17

| Constituent | Weight (g) |
|---|---|
| Paclitaxel | 1 |
| HPMC 2910 | 7 |
| Poloxamer 188 | 25 |
| Solutol | 0.75 |
| d-α-Tocopherol | 1 |
| Dichloromethane | 396 |
| Ethanol | 264 |

### EXAMPLE 18

### Preparation of a Highly Uniform Nanoscale Paclitaxel Solid Dispersion by Supercritical Fluid Process 18

A highly uniform nano-scale pacliitaxel solid dispersion for injection was prepared using the constituents shown in Table 18 according to the same supercritical fluid process as described in Example 1 except that Tween 80 and Flurorinc L-44 (BASF) were employed as a surfactant, and the supercritical operation was carried out under the condition of 40° C. and 136 bar.

TABLE 18

| Constituent | Weight (g) |
|---|---|
| Paclitaxel | 1 |
| HPMC 2910 | 7 |
| Tween 80 | 10 |
| Fluronic L-44 | 10 |
| Solutol | 0.75 |
| Myrj 52 | 10 |
| d-α-Tocopherol | 3 |
| Dichloromethane | 475 |
| Ethanol | 317 |

### EXAMPLE 19

### Preparation of a Highly Uniform Nano-Scale Paclitaxel Solid Dispersion by Supercritical Fluid Process 19

A highly uniform nano-scale pacliitaxel solid dispersion for injection was prepared using the constituents shown in Table 19 according to the same supercritical fluid process as described in Example 1 except that Cremophor RH40 (BASF) was employed as a surfactant, and the supercritical operation was carried out under the condition of 40° C. and 103 bar.

TABLE 19

| Constituent | Weight (g) |
|---|---|
| Paclitaxel | 1 |
| HPMC 2910 | 7 |
| Cremophor RH40 | 25 |
| Solutol | 0.75 |
| Ascorbil palmitate | 0.5 |
| d-α-Tocopherol | 1 |
| Dichloromethane | 400 |
| Ethanol | 268 |

### EXAMPLE 20

### Preparation of a Highly Uniform Nano-Scale Paclitaxel Solid Dispersion by Supercritical Fluid Process 20

A highly uniform nano-scale pacliitaxel solid dispersion for injection was prepared using the constituents shown in Table 20 according to the same supercritical fluid process as described in Example 1 except that Cremophor RH60 (BASF) was employed as a surfactant, and the supercritical operation was carried out under the condition of 40° C. and 103 bar.

TABLE 20

| Constituent | Weight (g) |
|---|---|
| Paclitaxel | 1 |
| HPMC 2910 | 7 |
| Cremophor RH60 | 30 |
| Solutol | 0.75 |
| Ascorbil palmitate | 0.5 |
| d-α-Tocopherol | 3 |
| Dichloromethane | 480 |
| Ethanol | 320 |

### EXAMPLE 21

### Preparation of a Highly Uniform Nano-Scale Paclitaxel Solid Dispersion by Supercritical Fluid Process 21

A highly uniform nano-scale pacliitaxel solid dispersion for injection was prepared using the constituents shown in Table 21 according to the same supercritical fluid process as described in Example 1 except that Cremophor RH60 (BASF) and Cremophor EL (BASF) were employed as a surfactant, and the supercritical operation was carried out under the condition of 40° C. and 103 bar.

TABLE 21

| Constituent | Weight (g) |
|---|---|
| Paclitaxel | 1 |
| HPMC 2910 | 7 |
| Cremophor RH40 | 30 |
| Cremophor EL | 5 |
| Solutol | 0.75 |
| Ascorbil palmitate | 0.5 |
| d-α-Tocopherol | 3 |
| Dichloromethane | 538 |
| Ethanol | 360 |

### EXAMPLE 22

### Preparation of a Highly Uniform Nano-Scale Paclitaxel Solid Dispersion by Supercritical Fluid Process 22

A highly uniform nano-scale pacliitaxel solid dispersion for oral was prepared using the constituents shown in Table 22 according to the same supercritical fluid process as described in Example 1 except that sodium lauryl sulfate was employed as a surfactant, and the supercritical operation was carried out under the condition of 40° C. and 115 bar.

TABLE 22

| Constituent | Weight (g) |
|---|---|
| Paclitaxel | 1 |
| HPMC 2910 | 5 |
| Sodium lauryl sulfate | 1 |
| Dichloromethane | 80 |
| Ethanol | 53 |

### EXAMPLE 23

### Preparation of a Highly Uniform Nano-Scale Paclitaxel Solid Dispersion by Supercritical Fluid Process 23

A highly uniform nano-scale pacliitaxel solid dispersion for oral was prepared using the constituents shown in Table 23 according to the same supercritical fluid process as described in Example 1 except that sodium lauryl sulfate was employed as a surfactant, and the supercritical operation was carried out under the condition of 60° C. and 103 bar.

TABLE 23

| Constituent | Weight (g) |
|---|---|
| Paclitaxel | 1 |
| HPMC 2910 | 3 |
| Sodium lauryl sulfate | 0.4 |
| Dichloromethane | 50 |
| Ethanol | 33 |

### EXAMPLE 24

### Preparation of Solid Dispersion Using Liquid Carbon Dioxide

The same supercritical fluid process as described in Example 1 except that sodium lauryl sulfate was employed as a surfactant, and the supercritical operation was carried under the condition of 25° C. and 60 bar was carried out to prepare a highly uniform nano-scale pacliitaxel solid dispersion for oral using the constituents shown in Table 24. However, it was failed to prepare a separately spherical solid dispersion due to the formation of particle's aggregation.

TABLE 24

| Constituent | Weight (g) |
|---|---|
| Paclitaxel | 1 |
| HPMC 2910 | 5 |
| Sodium lauryl sulfate | 1 |
| Dichloromethane | 80 |
| Ethanol | 53 |

### TEST EXAMPLE 1

### Measurement of Saturation Solubility of the Paclitaxel Solid Dispersion Prepared by Supercritical Fluid Process

To measure the saturation solubility of the paclitaxel solid dispersion prepared by the supercritical fluid process, the paclitaxel solid dispersions for injection prepared in Example 1 to 5, the paclitaxel solid dispersion for oral prepared in Example 22 and the solid dispersion prepared using an liquid carbon dioxide in Example 24 as an experimental sample and a conventional paclitaxel powder (Hanmi Pharm. Co., Ltd.) as a control sample were subjected to the following experiment.

A large quantity of a sample (about 3 mg based on the amount of paclitaxel) and 1 ml of distilled water were placed in an eppendorf tube and mixed for 5 hrs at room temperature by using a vortex (Glas-Col® multi-pulse vortex; motor 30, pulser 30). After the eppendorf tube was left at room temperature for about 24 hr, it was subjected to a filtration using 0.45 μm of a filter paper. The filtrate thus obtained was subjected to HPLC analysis to measure the amount of paclitaxel dissolved in the filtrate, and the results are shown in Table 25. The HPLC analysis was carried out with Hitachi L-7100 pump, Hitachi UV detector L-7400, Rheodyne 7725i injector and Inertsil ODS2 (C18) column (5 m, 4.6 mm×15 cm, GL Science), and the absorbance was measured at UV 228 nm. Further, 50% acetonitrile aqueous solution was employed as a mobile phase, and its flow rate and injection volume were 1 ml/min and 20 ml, respectively.

TABLE 25

| | Degree of saturated |
|---|---|
| Sample | solubility (ppm) |
| Example 1 | 1800 |
| Example 2 | 2000 |
| Example 3 | 1700 |
| Example 4 | 1800 |
| Example 5 | 2100 |
| Example 22 | 2500 |
| Example 24 | 30 |
| Control | 0.57 |

As shown in Table 25, the saturation solubilities of the paclitaxel solid dispersions prepared by the supercritical fluid process of the present invention are markedly higher than that of the solid dispersion prepared using liquid carbon dioxide or a conventional paclitaxel powder.

### TEST EXAMPLE 2

### DSC Analysis of the Paclitaxel Solid Dispersion Prepared by Supercritical Fluid Process

The thermochemical property of the paclitaxel solid dispersion prepared by the supercritical fluid process of Example 1 was measured by using a differential scanning calorimeter (DSC) as follows.

After weighing about 3 mg of a sample placed in an aluminum pan using a micro balance, the aluminum pan was sealed with a cap to be used as a test sample. An empty aluminum pan with its cap was used as a control. The thermal change was analyzed with a DSC (Rheometric Scientific, Model: DLOS) at a scanning rate of 10° C. per minute. The parclitaxel solid dispersion of Example 1 and a parclitaxel powder were employed as samples.

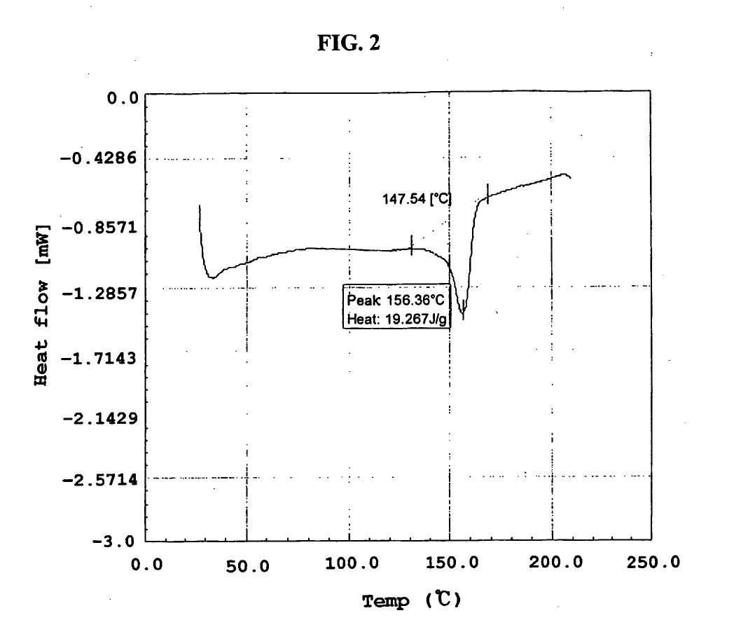

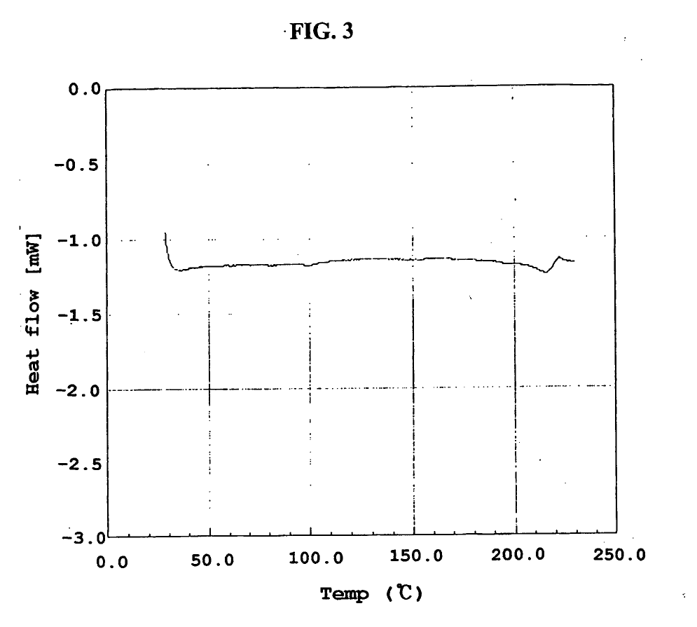

As a result, the paclitaxel powder showed a strong endothermic peak at around 156° C. as illustrated in FIG. 2, which is due to the paclitaxel's melting point being about 150° C. On the other hand, as shown in FIG. 3, the paclitaxel solid dispersion of the present invention did not show any endothermic peak. From these results, it has been found that the paclitaxel solid dispersion of the present invention is a highly uniform nano-scale particel having an altered molecular arrangement for a thermochemical property different from that of a conventional paclitaxel powder.

### TEST EXAMPLE 3

### SEM Analysis of the Paclitaxel Solid Dispersion Prepared by Supercritical Fluid Process

The shape of the paclitaxel solid dispersion prepared by the supercritical fluid process of Example 1 was examined by scanning electron microscopy (SEM) as follows.

The paclitaxel solid dispersion for injection of Example 1 and that for oral administration of Example 22 were each placed on a sample dish, and fixed thereto using a carbon adhesive. The sample dish was placed on a stage of an ion coating device and subjected to platinum coating for 2 to 3 min. Then, the paclitaxel solid dispersion was examined by SEM (FEI company, Model: XL30ESEMFEG) under 20 kV of voltage.

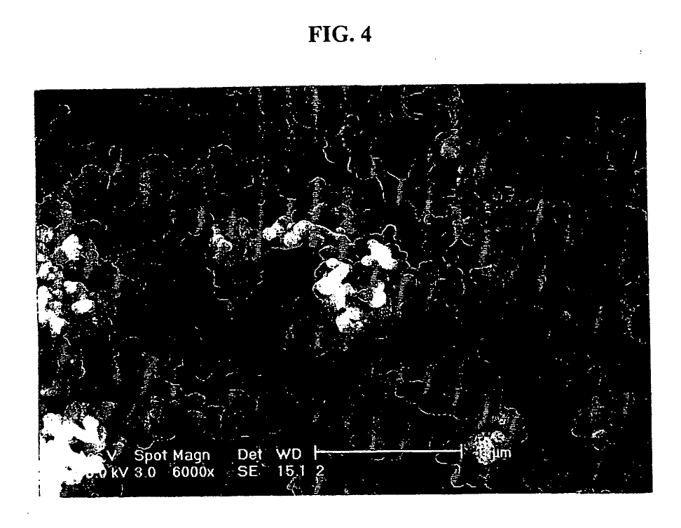

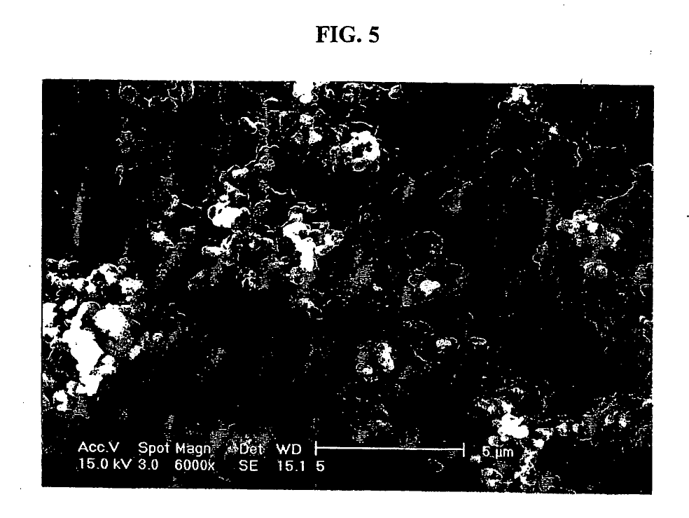

As a result, as illustrated in FIGS. 4 and 5, it has been found that the particles of the highly uniform nano-scale paclitaxel solid dispersion were of 1 μm or less in diameter.

1. A method for the preparation of a highly uniform nano-scale paclitaxel solid dispersion by a supercritical fluid process which comprises:
1) preparing a mixture of paclitaxel and a pharmaceutically acceptable additive and dissolving in a mixed organic solvent to obtain a solution mixture;
2) forming particles of the mixture of paclitaxel and the pharmaceutically acceptable additive by spraying the solution mixture of Step 1) to the supercritical fluid to bring into contact with each other;
3) removing the organic solvent by washing the particles with a fresh batch of the supercritical fluid; and
4) recovering the particles prepared thereby.

2. The method of claim 1, wherein the additive is a hydrophilic polymer or a surfactant.

3. The method of claim 2, wherein the hydrophilic polymer is one or more selected from the group consisting of hydroxypropylmethylcellulose (HPMC), polyvinylpyrrolidone, hydroxypropylcellulose (HPC), hydroxyethylcellulose (HEC) and Eudragit.

4. The method of claim 2, wherein the hydrophilic polymer is employed in an amount ranging from 0.1 to 20 weight part based on 1 weight part of paclitaxel.

5. The method of claim 2, wherein the amount of the hydrophilic polymer in the solution as a solvent-free basis is in the range of 1 to 75% (w/w).

6. The method of claim 1, wherein the mixed organic solvent is prepared by mixing two organic solvents, one being capable of dissolving paclitaxel and the other being capable of dissolving the additives.

7. The method of claim 6, wherein the two organic solvents are mixed in a weight ratio ranging from 7:3 to 5:5.

8. The method of claim 6, wherein the organic solvent for dissolving paclitaxel is selected from the group consisting of dichloromethane, chloroform, carbon tetrachloride, ethylacetate, N,N-dimethylformamide, dimethylsulfoxide and tetrahydrofuran.

9. The method of claim 6, wherein the organic solvent for dissolving the additive is selected from the group consisting of ethanol, methanol and isopropanol.

10. The method of claim 1, the supercritical fluid is contacted with the solution mixture containing paclitaxel and the additive under the condition of 35 to 70° C. and 80 to 200 bar.

11. A paclitaxel solid dispersion prepared by one of the the method of claim 1.

12. The paclitaxel solid dispersion of claim 11, which shows a thermochemical property determined by differential scanning calorimeter (DSC) different from that of a paclitaxel powder.

13. A pharmaceutical composition of paclitaxel for oral and injection administration, which comprises the paclitaxel solid dispersion of claim 11 as an effective ingredient.
